(19) United States
(12) Patent Application Publication
(10) Pub. No.: US 2021/0196177 A1
(43) Pub. Date: Jul. 1, 2021

(54) MAGNETOENCEPHALOGRAPHY

(71) Applicant: X Development LLC, Mountain View, CA (US)

(72) Inventors: Guillaume Verdon-Akzam, Mountain View, CA (US); Xudong Lv, Albany, CA (US); Stefan Leichenauer, Mountain View, CA (US)

(21) Appl. No.: 17/138,696

(22) Filed: Dec. 30, 2020

Related U.S. Application Data
(60) Provisional application No. 62/955,728, filed on Dec. 31, 2019.

(51) Int. Cl. A61B 5/245; A61B 5/00

(52) U.S. Cl. CPC A61B 5/245; A61B 5/6803; A61B 2562/0223; A61B 5/6868; A61B 5/6848

(57) ABSTRACT

A magnetoencephalography apparatus includes: a lead configured to be secured to a user's head; a first magnetic field sensor attached to the lead, the first magnetic field sensor including a substrate, and an electron spin defect layer on the substrate, the electron spin defect layer including at least one lattice defect, in which a first spin energy level of the at least one lattice defect splits upon exposure to a microwave; and cabling, in which the cabling includes a first microwave transmission line arranged to provide a first microwave field to the electron spin defect layer and in which the cabling includes an optical fiber arranged to provide, from a first end of the optical fiber, a first light signal to the electron spin defect layer and to receive, at the first end of the optical fiber, a second light signal emitted by the electron spin defect layer.

### CROSS-REFERENCE TO RELATED APPLICATION

This application claims the benefit of the filing date of U.S. Provisional Application No. 62/955,728, filed on Dec. 31, 2019. The contents of U.S. Provisional Application No. 62/955,728 are incorporated herein by reference in their entirety.

### BACKGROUND

Magnetoencephalography includes neuroimaging for mapping brain activity by recording magnetic fields produced by electrical currents occurring naturally in the brain.

### SUMMARY

The present disclosure relates to magnetoencephalography.

In general, in some aspects, the present disclosure encompasses a magnetoencephalography (MEG) apparatus that includes: a lead configured to be secured to a head of a user; a first magnetic field sensor attached to the lead, the first magnetic field sensor including a substrate, and an electron spin defect layer on the substrate, the electron spin defect layer including at least one lattice defect, in which a first spin energy level of the at least one lattice defect splits upon exposure to a microwave field; and cabling coupled to the lead, in which the cabling includes a first microwave transmission line arranged to provide a first microwave field to the electron spin defect layer and in which the cabling includes an optical fiber arranged to provide, from a first end of the optical fiber, a first light signal to the electron spin defect layer and to receive, at the first end of the optical fiber, a second light signal emitted by the electron spin defect layer.

Implementations of the MEG apparatus may include one or more of the following features. For example, in some implementations, the electron spin defect layer includes diamond. The at least one lattice defect may include a nitrogen vacancy defect.

In some implementations, the first magnetic field sensor further includes a bias magnet.

In some implementations, the MEG apparatus further includes an optical light source arranged to output the first light signal into a second end of the optical fiber. The first light signal may be a light signal having a wavelength of approximately 532 nm. The optical light source may be a light emitting diode or a laser. The MEG apparatus may further include a photodetector arranged to receive the second light signal from the second end of the optical fiber and to generate an optical measurement signal responsive to detecting the second light signal. The photodetector may be configured to detect light having a wavelength of approximately 630 nm. The MEG apparatus may further include an analog-to-digital converter coupled to the photodetector to receive the optical measurement signal. The MEG apparatus may further include a microprocessor, in which the microprocessor is configured to analyze the optical measurement signal to determine characteristics of a magnetic field to which the MEG is exposed.

In some implementations, the MEG apparatus may further include a microwave field generator coupled to an end of the microwave transmission line and configured to generate a first microwave field. The MEG apparatus may further include a microwave field control circuit coupled to the microwave field generator to provide the microwave field generator with a microwave source signal, in which the microwave field control circuit is optionally configured to output the microwave source signal at a frequency between about 50 MHz and about 4 GHz.

In some implementations, the MEG apparatus further includes multiple additional leads and multiple additional magnetic field sensors attached to the multiple additional leads, respectively, in which each additional magnetic field sensor of the multiple additional magnetic field sensors includes a corresponding substrate, and a corresponding electron spin defect layer on the substrate of the additional magnetic field sensor, the corresponding electron spin defect layer including at least one corresponding lattice defect, in which a first spin energy level of the at least one corresponding lattice defect splits upon exposure to a corresponding microwave field.

In some implementations, the MEG apparatus includes a cranial cap, in which the lead is attached to the cranial cap.

In general, in some other aspects, the subject matter of the present disclosure may be embodied in an intracranial magnetoencephalography (MEG) device that includes: a base; a first intracranial needle including a first end attached to the base, in which the first intracranial needle comprises a first microwave transmission line and a first optical waveguide; and a magnetic field sensor attached to a second end of the first intracranial needle, in which the magnetic field sensor includes a substrate, and an electron spin defect layer on the substrate, the electron spin defect layer including at least one lattice defect, in which a first spin energy level of the at least one lattice defect splits upon exposure to a microwave field.

Implementations of the intra-cranial MEG may include one or more of the following features. For example, in some implementations, the first microwave transmission line is arranged to provide a first microwave field to the electron spin defect layer and the optical waveguide is arranged to provide a first light signal to the electron spin defect layer and to receive a second light signal emitted by the electron spin defect layer.

In some implementations, the base includes an optical light source positioned to provide light into the optical waveguide. The optical light source may include a light emitting diode or a laser. The optical light source may be configured to emit a first light signal having a wavelength of approximately 532 nm.

In some implementations, the base includes a photodetector positioned to receive light from the optical waveguide and to generate an optical measurement signal responsive to detecting the second light signal. The photodetector may be configured to detect light having a wavelength of approximately 630 nm. The base may further include an analog-to-digital converter coupled to the photodetector to receive the optical measurement signal. The base may include a microprocessor configured to analyze the optical measurement signal to determine characteristics of a magnetic field to which the MEG is exposed. The base may include a transceiver configured to emit and receive wireless signals.

In some implementations, the base includes a microwave field generator configured to generate a first microwave field, in which the microwave field generator is coupled to the first microwave transmission line. The base may further include a microwave field control circuit coupled to the microwave field generator to provide the microwave field generator with a microwave source signal, in which the microwave field control circuit is optionally configured to output the microwave source signal at a frequency between about 50 MHz and about 4 GHz.

In some implementations, the first optical waveguide includes an optical fiber.

In some implementations, the electron spin defect layer includes diamond. The at least one lattice defect may include a nitrogen vacancy defect.

In some implementations, the magnetic field sensor further includes a bias magnet.

The details of one or more embodiments are set forth in the accompanying drawings and the description below. Other features and advantages will be apparent from the description and drawings, and from the claims.

### BRIEF DESCRIPTION OF THE DRAWINGS

FIG. 1 is a schematic that illustrates an example of an energy level diagram for a defect layer of a magnetic field sensor.

FIG. 2 is a schematic that illustrates an example of a magnetic field sensor.

FIG. 3 is a schematic that illustrates an example of a extra-cranial magnetoencephalography apparatus.

FIG. 4A-4D are schematics that illustrate examples of intra-cranial magnetoencephalography devices.

### DETAILED DESCRIPTION

The present disclosure relates to magnetoencephalography. In a particular example, the present disclosure relates to quantum sensors for detecting magnetic fields generated from the brain. In another more particular example, the present disclosure relates to electron spin defect based magnetometry for detecting magnetic fields generated from the brain. The magnetic field detection techniques disclosed herein rely, in part, on monitoring Zeeman shift of electron spin sublevels established by the presence of atomic defects in solid-state lattice structures.

More specifically, electron spin defect based magnetometers include quantum sensors that leverage the occurrence of an electronic spin defect in a solid state lattice, where the spin can be both initialized and read out optically. In certain implementations, the defect may arise as an atomic-level vacancy in a lattice structure, such as a vacancy occurring near a nitrogen atom substituted in place of a carbon atom within diamond. Accordingly, a single spin defect center, as an atom-scale defect, may be used to detect magnetic fields with nanometer spatial resolution, while an ensemble of spin defects may be used with spatial resolution given by the ensemble size (e.g., on the order of microns) typically with an improvement in sensitivity given by √N, where N is the number of spin defects. Moreover, in some implementations, electron spin defect based magnetometers may exhibit relatively long coherence times, such as times approaching 1 second or more. Additionally, electron spin defect based magnetometers may be operated at room temperature and, in certain cases, within relatively compact structures, allow for portability and reduction in magnetometer costs, which may be advantageous in health related applications such as measuring magnetic fields emanating from the brain.

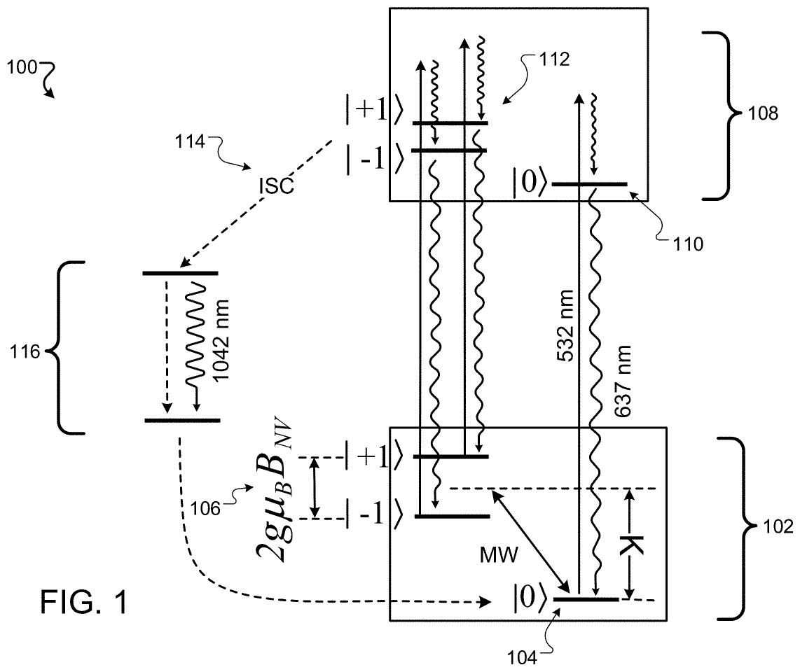

A brief description of electron spin defect based magnetometry will be described with reference to FIG. 1 and in particular with respect to nitrogen vacancy (NV) magnetometry, though the techniques and devices disclosed herein may be applicable to other materials, including other types of electron spin defects, as well. An NV center is a defect in a diamond lattice that contains a substitutional nitrogen atom in place of carbon, adjacent to a vacancy in the diamond lattice. The negatively-charged state of the defect provides a spin triplet ground level which can be initialized, coherently manipulated with long coherence time and readout, using optical means. FIG. 1 is a schematic that illustrates an energy level scheme 100 for an NV defect. The NV defect behaves as an artificial atom within the diamond lattice that exhibits a broadband photoluminescence emission with a zero phonon line at 1.945 eV or λ_{PL}=637 nm. Moreover, the ground level 102 of the NV defect is a spin triplet state, having spin sub-levels of the mₛ=0 state 104 and the mₛ=+/−1 states 106, separated by K=2.87 GHz in the absence of a magnetic field. The defect can be optically excited to an excited level 108, which also is a spin triplet having an mₛ=0 state 110 and mₛ=+/−1 states 112. Once optically excited into the excited level 108, the NV defect can relax primarily through one of two mechanisms: a) through a radiative transition and phonon relaxation, thus producing a broadband red photoluminescence; or b) through a secondary path 114 that involves non-radiative intersystem crossing (ISC) to singlet states 116.

The decay path branching ratios from the excited state manifold back to the ground state manifold depends on its initial spin sublevel projection. Specifically, if the electron spin began in the mₛ=+/−1 states, there is approximately a 30% chance for the spin to decay non-radiatively through the secondary path 114, down to the mₛ=0 state. The population of the spin sublevels can be manipulated by the application of a resonant microwave field to the diamond. Specifically, at a particular microwave frequency corresponding to the transition energy cost between the 0 and +/−1 states, transitions occur between those sublevels, resulting in a change in the level of photoluminescence of the system. In particular, if the spin is initialized into the ms=0 state, and the population is transferred to one of the +/−1 states by the resonant microwave drive, the photoluminescence rate upon subsequent optical illumination will decrease.

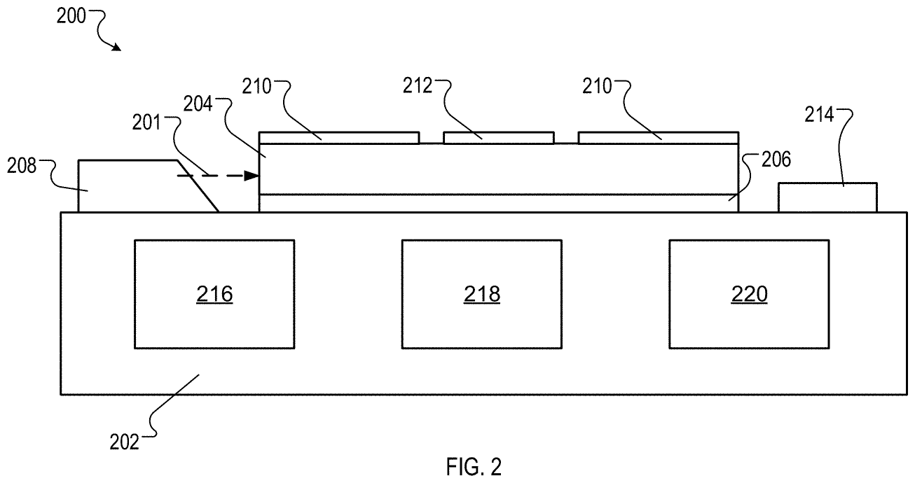

In the absence of a magnetic field, this drop in photoluminescence may be observed by sweeping the microwave frequency, as depicted in the bottom-most photoluminescence (PL) intensity line 202 shown in FIG. 2, which is a plot of PL intensity versus applied microwave frequency. Upon applying a magnetic field in the vicinity of the NV defect, however, the degeneracy of the mₛ=+/−1 spin sublevels is lifted by the Zeeman effect, leading to the appearance of two electron spin resonance (ESR) transitions, corresponding to dips in the PL spectrum (see upper PL lines 204 in FIG. 2). The value Av corresponds to the ESR linewidth, typically on the order of 1 MHz and the value C is the ESR contrast. To detect small magnetic fields, the NV transitions may be driven at the point of maximum slope (see, e.g., 206 in FIG. 2). At this point of maximum slope, a time-domain change in the photoluminescence may be detected, from which a time-domain change in magnetic field can be derived. The signal may be expressed as (∂I₀/∂B)×δB×Δt, where I₀ is the NV defect PL rate, δB is the infinitesimal magnetic field variation, and Δt is the measurement duration, much smaller than the timescale on which the magnetic field changes A single NV defect therefore can serve as a magnetic field sensor with an atomic-sized detection volume. To improve sensitivity, a collective response of an ensemble of NV defects may be detected, such that the collected PL signal is magnified by the number N of the sensing spins and therefore improves the shot-noise limited magnetic field sensitivity by a factor of 1/√N.

As explained above, an NV defect is just one example of a type of spin defect that may be used to perform electron spin defect based magnetometry. In other implementations, one or more spin defects may be formed in silicon carbide. SiC defects include defects due to other substitutional atoms, such as, e.g. phosphorus, in the SiC lattice. Similar techniques for detecting magnetic fields as described herein with NV defects may be employed with the SiC defects.

FIG. 2 is a schematic that illustrates an example of a device 200 that may be used to perform electron spin defect based magnetometry, as described herein. Device 200 includes a substrate 202 and an electron spin defect layer 204 formed on the substrate 202. The electron spin defect layer 204 may include multiple lattice point defects, such as NV defects formed in diamond, as described herein. The defect layer 204 containing the NV defects may be formed, in some cases, from up to 99.999% carbon 12. In some implementations, carbon 13 may be used partially in place of carbon 12. The electron spin defect layer 204 is not limited to NV defects formed in diamond, which is typically electronic grade, and may include other lattice point defects in other materials, such as silicon carbide. The electron spin defect layer 204 may be a sub-layer of a base layer 206 that is without the electron spin defects. For example, base layer 206 may be a diamond layer without NV defects, whereas a top portion of the diamond layer corresponds to the defect layer 204. The dimensions (e.g., thicknesses) of the different layers and objects depicted in FIG. 2 are not to scale.

The thickness of the defect layer 204 may vary. For example, in some implementations, the thickness of the defect layer 204 may be greater than about 2-3 microns, such as greater than 10 microns, greater than 50 microns, greater than 100 microns, greater than 250 microns, greater than 500 microns, or greater than 750 microns. The thickness of the defect layer 204 may be less than about 1 millimeter, such as less than 750 microns, less than 500 microns, less than 250 microns, or less than 100 microns. Other thicknesses may be used as well. Thickness of layer 204 is referenced here as being the distance from the interface between layer 204 and layer 206 and the opposite facing surface of layer 204. If the defect layer is a part of or formed on layer 206, then layer 206 may have its own separate thickness. For example, layer 206 may have a thickness between about 200 microns and about 5 millimeters. Thickness of layer 206 is referenced here as being the distance from the interface between layer 204 and layer 206 and the interface between layer 206 and substrate 202.

In some implementations, the layer 204 (or the layer 206) is secured to the substrate using an adhesive including, e.g., epoxies, elastomers, thermoplastics, emulsions, and/or thermosets, among other types of adhesives. In some implementations, electrical contacts are formed between the layer 204 (or the layer 206) and the substrate 202. For example, in some cases, the substrate may include a semiconductor material, such as silicon, in which one or more circuit elements (216, 218, 220) are fabricated. Electrical connections may be formed within the substrate 202 to provide an electrical connection among the circuit elements 216, 218, 220 and/or to one or more components formed in or on layer 204 (and/or layer 206).

Device 200 further includes a microwave field transmitter 210 to provide a microwave field to the defect layer 204. In the present example shown in FIG. 2, microwave field transmitter 210 includes a thin film antenna formed on an upper surface of the defect layer 204. In some implementations, the microwave field transmitter 210 includes a patterned layer of metal on a surface of the defect layer 204, within layer 206, at the interface between defect layer 204 and layer 206, or as a separate component held adjacent to layer 204. The microwave field transmitter 210 may include a co-planar waveguide, a wire, a loop or a coil of electrically conductive material, such as metal. The microwave field transmitter 210 may be positioned adjacent to the area of the defect layer 204 to which the light from an optical source is directed.

In some implementations, a microwave field control circuit 216, also referred to as a microwave field generator, provides a microwave source signal to the microwave field transmitter 210. The microwave field control circuit 216 may be formed as part of device 200, e.g., the microwave field control circuit 216 may be formed in or on the substrate 202. For example, in some implementations, the microwave control circuit 216 may be a circuit element formed within a silicon substrate. Alternatively, the microwave field control circuit 216 is separate from device 200 and is electrically connected to the field transmitter 210 of device 210 through cabling or wiring. The microwave control circuit 216 may be coupled, e.g., directly electrically connected, to the microwave field transmitter 210 to provide a microwave source signal to the microwave field transmitter 210 so that the microwave field transmitter 210 emits a microwave field toward the defect layer 204. The microwave source signal may optionally be a pulsed microwave source signal. In some implementations, a microwave frequency of the microwave source signal is between about 2 GHz and about 4 GHz. In some implementations, the microwave field transmitter 210 emits signals at multiple frequencies spaced apart from one another to drive additional energy level splittings. For example, in some implementations, the microwave field transmitter 210 may be operated to emit microwave signals that address NV hyperfine transitions. In some implementations, the microwave control circuit 216 is configured to provide a control signal that generates a pulsed microwave signal at the transmitter 210. In some implementations, the microwave control circuit 216 is configured to provide a control signal that generates a continuous wave microwave signal at the transmitter 210. Examples of microwave sources include, but are not limited to, RF signal generators available from Stanford Research Systems and waveform generators available from Tektronix.

In some implementations, the device 200 includes a photodetector 212 arranged to detect photoluminescence emitted from the electron spin defects of the defect layer 204. The photoluminescence may include one or more wavelengths of light, such as wavelengths of approximately 630 nm, including 637 nm, corresponding to the emission wavelength of an NV defect. The photodetector 212 may be positioned on an upper surface of the defect layer 204 and in direct contact with the defect layer 404 as shown in FIG. 2. In some implementations, the photodetector 212 is positioned so that a detecting surface of the photodetector 212 faces an area of the defect layer 204 to which the light from an optical source is directed. The photodetector 212 may be secured to the defect layer 204 using an adhesive that is optically transparent to the wavelengths of light emitted by the NV defects. Alternatively, or in addition, the photodetector 212 may be formed beneath defect layer 204, such as at an interface between substrate 202 and layer 204 or within substrate 202. For example, in some implementations, the photodetector 212 may be a silicon based photodetector formed within the substrate 202. In some implementations, an optical component is positioned between the photodetector 212 and the defect layer 204. For example, the optical component may include one or more of a lens, a beam-splitter, a diffraction grating, an optical filter, and/or a mirror. The optical filter may be configured to filter out wavelengths of light different than the wavelength of light emitted by the defects of the defect layer 204. In some implementations, the photodetector 212 may be held in place adjacent to the defect layer 204. For instance, the photodetector 212 may be secured within a case in which the defect layer 204 also is provided, and arranged to detect light emission from the defect layer 204. In some implementations, the device 200 includes multiple photodetectors, such as a photodiode array. The photodetectors 212 may be located at multiple different positions around the defect layer 404 in order to maximize collection of light emitted by the defect layer 204.

In some implementations, the device 200 includes a microprocessor 218, in which the microprocessor 218 is coupled to the photodetector 212 to receive a light measurement signal from the photodetector and in which the microprocessor is configured to analyze the light measurement signal to determine the characteristics of a magnetic field to which the device 200 is exposed. The microprocessor 218 may be formed in or on the substrate 202. For example, in some implementations, the microprocessor 218 may be a circuit element formed within a silicon substrate. The microprocessor 218 may be coupled, e.g., directly electrically connected, to the photodetector 212. Though the microprocessor 218 is depicted as being formed in the substrate 202, the microprocessor 218 may be located remotely from the device 200. For example, in some implementations, the device 200 may include a wireless transmitter/wireless receiver (i.e., a transceiver) to wirelessly receive control and analysis signals from the microprocessor 218 and to wirelessly transmit feedback and measurement data to the microprocessor 218. Alternatively, the microprocessor 218 is separate from the device 200 but coupled to the device 200 through, e.g., cabling, to send data to and receive data from the device 200.

In some implementations, the device 200 includes an optical source 208 configured to emit light. The light emitted by the optical source 208 may include a first wavelength that excites the one or more lattice point defects within the defect layer 204 from a ground state to an excited state. The first wavelength is different from a second wavelength that is emitted by the lattice point defects upon relaxation. The first wavelength may be, e.g., about 532 nm to excite NV defects in the defect layer 204. The optical source 208 may include, e.g., a light emitting diode, a laser, or a broadband source that includes filters configured to block transmission of wavelengths other than those used to excite the lattice point defects. The optical source 208 may be arranged to emit light 201 toward the defect layer 204. For example, the optical source 208 may be angled so that light 201 exiting the source 208 travels a path toward the defect layer 204. Alternatively, one or more optical elements may be positioned in front of the light emitted from the source 208 to redirect the light toward the defect layer 204. For example, the one or more optical components may include a lens, a mirror, a beam splitter, and/or a diffraction grating.

In some implementations, the device 200 includes an optical source circuit, e.g., a driver 220 for the optical source, in which the driver 220 is coupled to the optical source 208 to provide a control signal to drive the optical source. The driver 220 may be formed in or on the substrate 202. For example, in some implementations, the driver 220 may be a circuit element formed within a silicon substrate. The driver 220 may be coupled, e.g., directly electrically connected, to the optical source 208. In some implementations, the microprocessor 218 is coupled to one or both of the microwave field control circuit 216 and the driver 220 to control operation of the field control circuit 216 and/or the driver 220. In some implementations, the optical source 208 is separate from the device 200. For example, in some implementations, light from the optical source 208 may be delivered to the defect layer 204 of the device 200 using at least one optical waveguide, such as an optical fiber waveguide that is arranged to receive light output from the optical source 208 and emit the received light toward the defect layer 204. In some cases, the optical waveguide (or waveguides) used to deliver light to the defect layer 204 also may be used to transmit light emitted from the defect layer 204 to the photodetector 212. The optical waveguide(s) may be positioned facing one or more thin edges (thin relative to other edges) of the defect layer 204 to provide light to and/or detect light emitted from the defect layer 204. Alternatively, or in addition, the optical waveguide(s) may be positioned facing one or more large surface area sides (large relative to other sides) of the defect layer to provide light to and/or detect light emitted from the defect layer 204.

In some implementations, at least one optical component is arranged between the optical source 208 and the defect layer 204, so that the at least one optical component is positioned to direct the light from the optical source 208 through the defect layer 204 and towards an interface of the defect layer 204. In some implementations, the device 200 includes optical fibers, such as tapered optical fibers, for optically coupling light into and out of the defect layer 204.

In some implementations, the device 200 includes a magnet 214. The magnet 214 may be arranged adjacent to the electron spin defect layer 204. The magnet 214 is a bias magnet provided to induce the Zeeman effect and lift the degeneracy of the ms=+/−1 spin sublevels. In some implementations, the magnet 214 is a permanent magnet. The magnet 214 may be positioned directly on the substrate 202, on layer 206, or on layer 204, among other locations. For instance, the magnet 214 may be held in place adjacent to the defect layer 204 within a casing which also includes the defect layer 204. The magnet geometry may be chosen to minimize effects of inhomogeneous broadening between distinct defects in the defect layer 204.

A magnetic field sensor, such as the magnetic field sensors 200 described herein with respect to FIG. 2 may be used to detect magnetic fields from living things including, for example, magnetic fields emitted by animal brains. In turn, the detected magnetic fields may be analyzed to understand and interpret brain function. This functional neuroimaging technique may also be referred to as magnetoencephalography (MEG) and may be used to map brain activity by recording magnetic fields produced by electrical currents occurring naturally in the brain. In some cases even, the detected magnetic fields may be used as part of a process to write signals to the brain, e.g., to assist individuals with brain damage or having a disease that otherwise limits brain functionality. In general, though, electromagnetic signals from the brain and its constituent parts are relatively weak and subject to attenuation by intervening tissue, rendering detection of those fields difficult. Certain magnetic field detectors rely on extremely low temperatures to establish superconductivity and/or require substantial shielding to prevent interference from stray magnetic fields.

The magnetic field sensors of the present disclosure can be operated at room temperature, do not require elaborate shielding, and have the ability to accurately detect small fluctuations in weak magnetic fields. Moreover, the magnetic field sensors of the present disclosure can be made compact and low cost relative to more traditional MEG sensors such that they can be implemented as part of an extra-cranial apparatus or an intra-cranial device.

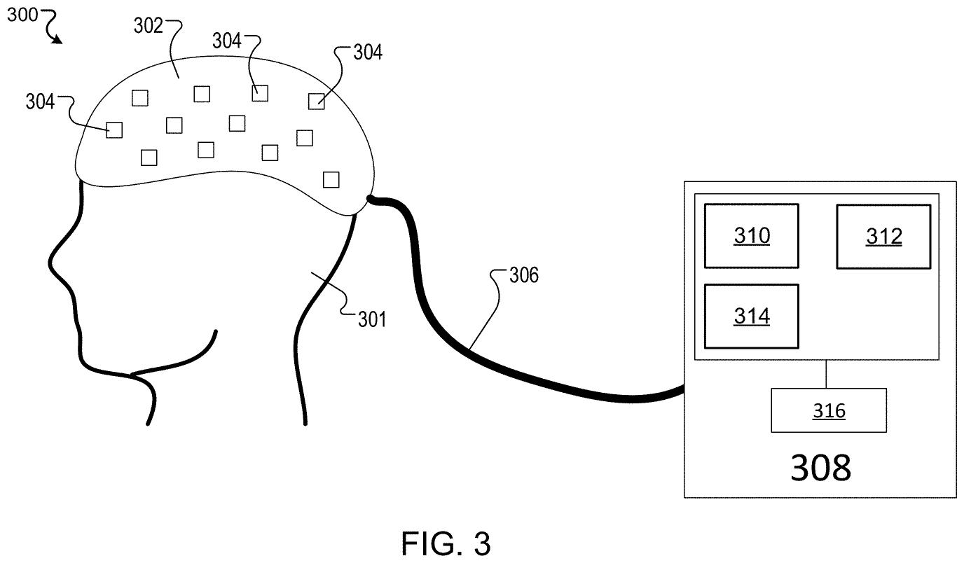

FIG. 3 is a schematic that illustrates an exemplary extra-cranial MEG apparatus 300 that uses one or more magnetic field sensors. The extra-cranial MEG apparatus 300 includes a cranial cap 302 to which are coupled one or more leads 304. Each lead 304 may include any of the magnetic field sensors disclosed herein, such as any of the magnetic field sensors described with respect to FIG. 2. The cranial cap 302 may be a cap that is shaped to fit around a head of a subject 301. The subject 301 may be a person or other animal. The cranial cap 302 may be implemented as, e.g., a piece of cloth, synthetic fiber, plastic, or rubber that is either fitted or expandable to fit different sized heads. In some implementations, the cap 302 may be a helmet, hat or other head gear for fitting around a user's head. The one or more leads 304 may be embedded in or fixed to a surface of the cranial cap 302.

Though shown embedded in the cranial cap 302, the cap 302 may be excluded from apparatus 300. For instance, the apparatus 300 may include one or more leads 304 not attached to or part of a cap. In such cases, the one or more leads 304 may be configured to attach to a user's head. For instance, the one or more leads 304 may include an adhesive, paste, gel or the like that allows the leads 304 to be removably attached to the skin of the user 301.

The apparatus 300 further includes cabling 306 coupled to the one or more leads 304. The cabling 306, in turn, is coupled to a data collection and processing module 308. In some implementations, the cabling 306 is coupled to each magnetic field sensor of each of the one or more leads 304. The cabling 306 may include at least one microwave transmission line. The at least one microwave transmission line may be arranged to provide a first microwave field to a corresponding magnetic field sensor of a lead 304. For instance, the microwave transmission line of the cabling 306 may be arranged so that a first end or loop is placed or positioned near a defect layer (e.g., defect layer 204 of device 200) so that a microwave field emitted from the transmission line is directed to the defect layer of the magnetic field sensor. A second end of the microwave transmission line of the cabling 306 may be coupled to a microwave source generator 310 of the data collection and processing module 308. The microwave transmission line may include, e.g., a coaxial waveguide. The microwave source generator 310 may be configured to output a microwave source signal having a frequency between about 50 MHz and about 4 GHz.

The cabling 306 may include at least one optical waveguide, such as an optical fiber (e.g., a wide-band optical fiber). The at least one optical waveguide may be arranged to provide light having a first wavelength to a corresponding magnetic field sensor of a lead 304. For instance, the optical waveguide of the cabling 306 may be arranged so that a first end of the waveguide is placed or positioned near a defect layer (e.g., defect layer 204 of device 200) so that light emitted from the optical waveguide is directed to the defect layer of the magnetic field sensor. The same or different optical waveguide (or waveguides) may also be positioned to receive light emitted from the defect layer 204. A second end of the optical waveguide of the cabling 306 may be coupled to an optical source 312, such as a light emitting diode or laser, of the data collection and processing module 308. The optical source 312 may be configured to emit light having a first wavelength that excites defects in a defect layer of the magnetic field sensor, e.g., about 532 nm to excite NV defects. The second end of the optical waveguide of the cabling 306 also may be coupled to a photodetector 314, such as a photodiode, contained within the data collection and processing module 308. For instance, a beam splitter may be positioned at the second end of the optical waveguide so that light exiting the waveguide is delivered to the photodetector 314 whereas light from the light source 312 is coupled into the waveguide. In some implementations, the photodetector 314 is configured to detect light having a particular wavelengths of light, such as wavelengths of approximately 630 nm, including 637 nm, corresponding to the emission wavelength of an NV defect.

As explained herein, the data collection and processing module 308 may include a microwave field source generator 310, an optical source 312, and/or a photodetector 314. The data collection and processing module 308 also may include a microprocessor 316. The microprocessor 316 may be coupled to one or more of the generator 310, the optical source 312 and/or the photodetector 314 to provide control signals and/or to receive data. The microprocessor 316 may receive a light measurement signal from the photodetector 314, in which the microprocessor is configured to analyze the light measurement signal to determine the characteristics of a magnetic field to which one or more magnetic field sensors of the apparatus 300 are exposed. In some implementations, the microprocessor 316 includes an analog-to-digital converter to convert the light measurement signal from the photodetector 314 into a digital signal. The microprocessor 316 may include a driver that provides an optical control signal to the optical source 312 to cause the optical source 312 to emit light.

Though a single microprocessor 316 is shown, the data collection and processing module 308 may include multiple microprocessors that together perform some or all of the microprocessor operations described herein. The microprocessor 316 may be a part of a computing system that includes associated memory on which is stored instructions executable by the microprocessor 316 to perform operations described herein. The microprocessor 316 may be configured to conjugate the signals received from the photodetector with spatial information of the location of the magnetic field sensors relative to the brain to construct a 2D or 3D intra-cortical magnetic field map. With further data processing, such as machine learning, of the signals received from the magnetic field sensors, it may be possible to develop a better understanding of the brain behavior. For example, the data may be relied on to understand neuron behaviors by analyzing the 2D or 3D magnetic field map at different locations of the brain. In some cases, the data may be used to understand neuron behaviors within other parts of the body other than brain by observing correlations between brain activity and neural responses within the other parts of the body, thus aiding in drawing connections between neuron activities and its consequences.

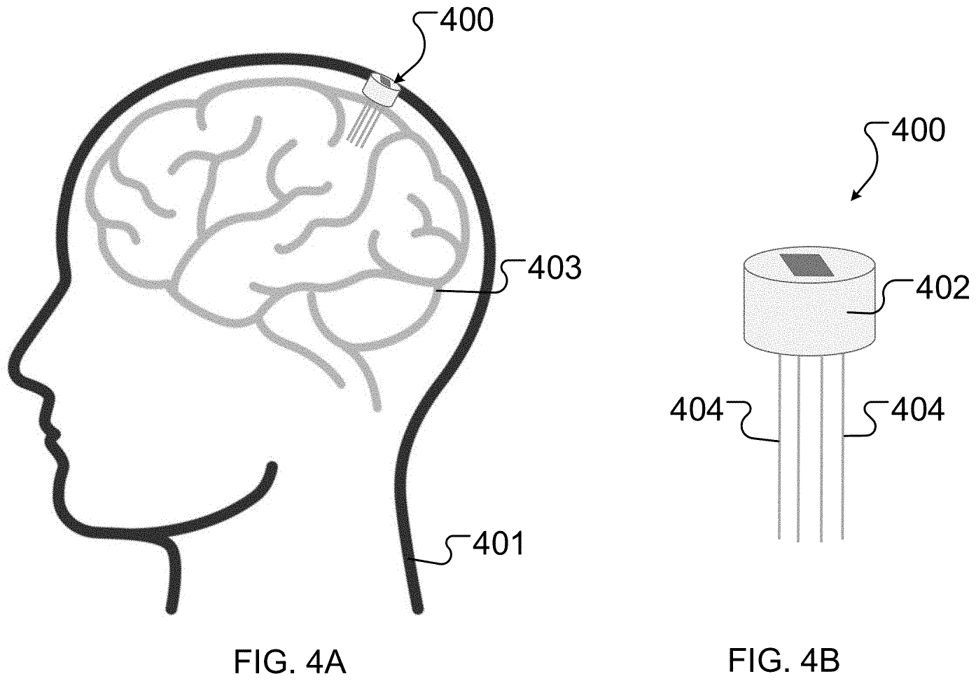

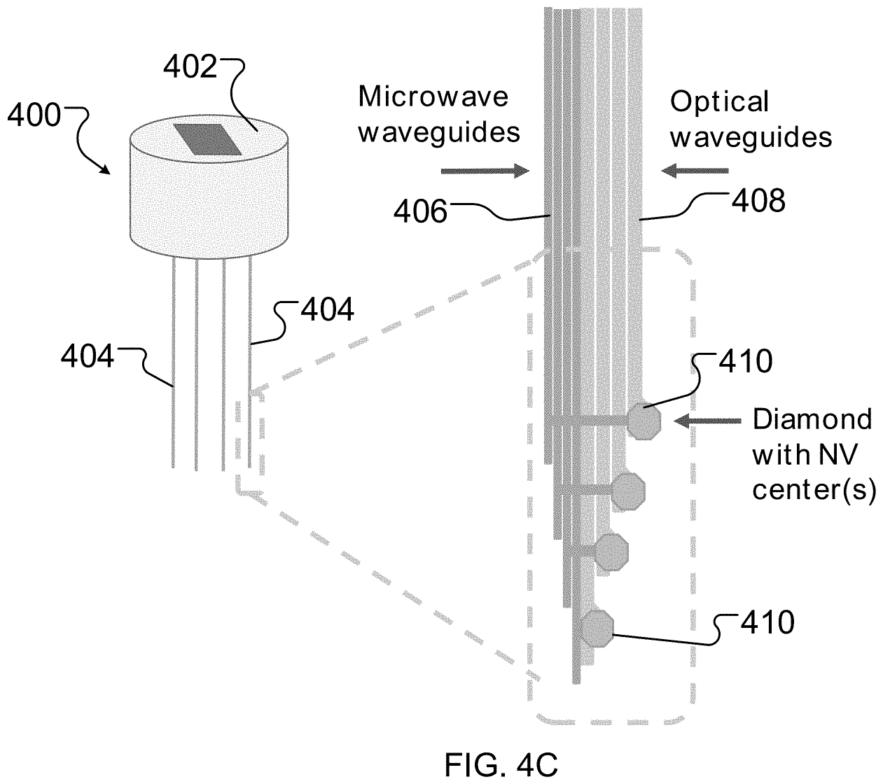

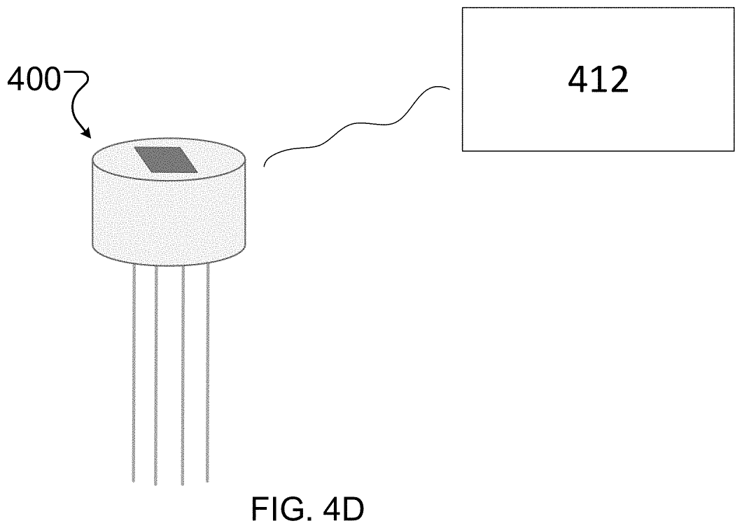

FIG. 4A is a schematic that illustrates an exemplary intra-cranial MEG device 400 that uses one or more magnetic field sensors. The intra-cranial MEG device 400 is designed to be inserted at least partially within brain tissue 403 of a subject 401. Given that the intra-cranial device is inserted into the brain tissue 403 directly, magnetic fields from the brain tissue 403 are likely to be stronger and more easily detected. FIG. 4B is a schematic illustrating a close-up of the intra-cranial device 400. The device 400 includes a base 402 and one or more intracranial needles 404 extending from the base 402. The intra-cranial needles 404, also referred to as subdural needles 404, are constructed to be inserted into brain tissue. For instance, the needles 404 are formed of a material hard enough such that the needles 404 do not deform upon insertion into the brain tissue. For instance, in some implementations, the needles 404 are constructed from sterile, disposable stainless steel, platinum, gold, though other materials, such as copper or silicon, may be used instead. For instance, to prevent shielding that would otherwise prevent detection of a desired magnetic field, the needles 404 may be formed from a material that does not conduct electromagnetic fields, such as plastic.

A first end of each intra-cranial needle 404 is coupled to the base 402, whereas a second opposite end or tip of each intra-cranial needle 404 is provided for insertion into brain tissue. In some implementations, the intra-cranial needles 404 are constructed as hollow cylinders. Alternatively, the intra-cranial needles 404 have solid cores. A needle 404 may carry a microwave transmission line and/or an optical waveguide, such as the microwave transmission lines and optical waveguides described herein, through its hollow or solid core. In some cases, a needle 404 carries multiple microwave transmission lines and/or optical waveguides through its hollow or solid core. In some implementations, the microwave transmission line and optical waveguide extend fully from the first end near the base 402 through the core to the second end of the needle 404 near the needle tip. Alternatively, the microwave transmission line and optical waveguide extend only partially the way through the core, e.g., from the first end near the base 402 to half-way through or three-quarters way through the core towards the needle tip. In some implementations, each intra-cranial MEG device 400 includes multiple needles 404 coupled to the base 402.

A close-up of the microwave transmission lines 406 and optical waveguides 408 within an intra-cranial needle 404 is shown in FIG. 4C. A magnetic field sensor 410 is provided at the end of a corresponding microwave transmission line 406 and a corresponding optical waveguide 408. The magnetic field sensor 410 may include any of the magnetic field sensors disclosed herein, such as any of the magnetic field sensors described with respect to FIG. 2. The magnetic field sensor 410 may be attached to the ends of the microwave transmission line 406 and the optical waveguide 408. For instance, in some implementations, the ends of the microwave transmission line 406 and the optical waveguide 408 may be bonded to the magnetic field sensor 410 using a bonding or epoxy adhesive. The end of the microwave transmission line 406 that is attached to the magnetic field sensor 410 may be arranged so that the line 406 can provide a microwave field to a defect layer of the magnetic field sensor 410. For instance, the microwave transmission line 406 may be arranged so that a first end or loop is placed or positioned near a defect layer (e.g., defect layer 204 of device 200) of the sensor 410 so that a microwave field emitted from the transmission line is directed to the defect layer of the magnetic field sensor 410. The end of the optical waveguide 408 that is attached to the magnetic field sensor 410 may be arranged so that the waveguide 408 can provide input light to the defect layer of the magnetic field sensor 410 and receive light emitted by the defect layer of the magnetic field sensor 410. In some cases, as shown in FIG. 4C, multiple magnetic field sensors 410 are included within a core of or as part of an intra-cranial needle 404. Each sensor 410 then may be coupled to a corresponding microwave transmission line 406 and a corresponding optical waveguide 408.

In some implementations, the base 402 includes an optical source, such as a light emitting diode or laser. The optical source of the base 402 is coupled to one or more optical waveguides 408 of the intra-cranial needles 404 so as to provide input light into the waveguides 408. The light emitted by the optical source may include a first wavelength that excites the one or more lattice point defects within the defect layer of the magnetic field sensor 410, such as light having a wavelength of about 532 nm.

In some implementations, the base 402 includes a photodetector. The photodetector of the base 402 is coupled to one or more optical waveguides 408 of the intra-cranial needles 404 so as to receive light from the waveguides 408. The photodetector of the base 402 may be configured to detect light having a particular wavelengths of light, such as wavelengths of approximately 630 nm, including 637 nm, corresponding to the emission wavelength of the defect layer in the magnetic field sensor.

In some implementations, the base 402 includes a microwave source generator. The microwave source generator may be configured to output a microwave source signal having a frequency between about 50 MHz and about 4 GHz. The microwave source signal may be coupled to the microwave transmission line that extends through the intra-cranial needle.

In some implementations, the base 402 also includes at least one microprocessor. The at least one microprocessor may be coupled to one or more of the microwave source generator, the optical source and/or the photodetector of the base to provide control signals and/or to receive data. The at least one microprocessor of the base 402 may receive a light measurement signal from the photodetector in the base 402, in which the microprocessor is configured to analyze the light measurement signal to determine the characteristics of a magnetic field to which one or more magnetic field sensors of the device 400 are exposed. In some implementations, the at least one microprocessor includes an analog-to-digital converter to convert the light measurement signal from the photodetector of the base 402 into a digital signal. The at least one microprocessor of the base 402 may include a driver that provides an optical control signal to the optical source of the base 402 to cause the optical source to emit light.

In some implementations, the base 402 includes a wireless transmitter to emit wireless signals and a wireless receiver to receive wireless signals (e.g., a wireless transceiver). The at least one microprocessor may be provided in a data collection and processing apparatus 412 that is remote from the intra-cranial MEG device 400 and that is capable of wirelessly sending and receiving signals 414 from the transceiver of the base 402, as shown in FIG. 4D. The data collection and processing module 412 may be configured in the same manner as module 308. As explained herein, the at least one microprocessor may be configured to conjugate the signals received from the photodetector with spatial information of the location of the magnetic field sensors relative to the brain to construct a 2D or 3D intra-cortical magnetic field map. With further data processing, such as machine learning, of the signals received from the magnetic field sensors, it may be possible to develop a better understanding of the brain behavior. For example, the data may be relied on to understand neuron behaviors by analyzing the 2D or 3D magnetic field map at different locations of the brain. In some cases, the data may be used to understand neuron behaviors within other parts of the body other than brain by observing correlations between brain activity and neural responses within the other parts of the body, thus aiding in drawing connections between neuron activities and its consequences.

Operations performed by the data collection and processing modules disclosed herein can include operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A magnetoencephalography (MEG) apparatus comprising:
a lead configured to be secured to a head of a user;
a first magnetic field sensor attached to the lead, the first magnetic field sensor comprising
a substrate, and
an electron spin defect layer on the substrate, the electron spin defect layer comprising at least one lattice defect, wherein a first spin energy level of the at least one lattice defect splits upon exposure to a microwave field; and
cabling coupled to the lead, wherein the cabling comprises a first microwave transmission line arranged to provide a first microwave field to the electron spin defect layer and wherein the cabling comprises an optical fiber arranged to provide, from a first end of the optical fiber, a first light signal to the electron spin defect layer and to receive, at the first end of the optical fiber, a second light signal emitted by the electron spin defect layer.

2. The MEG apparatus of claim 1, wherein the electron spin defect layer comprises diamond.

3. The MEG apparatus of claim 2, wherein the at least one lattice defect comprises a nitrogen vacancy defect.

4. The MEG apparatus of claim 1, wherein the first magnetic field sensor further comprises a bias magnet.

5. The MEG apparatus of claim 1, further comprising an optical light source arranged to output the first light signal into a second end of the optical fiber.

6. The MEG apparatus of claim 5, wherein the first light signal is a light signal having a wavelength of approximately 532 nm.

7. The MEG apparatus of claim 5, wherein the optical light source is a light emitting diode or a laser.

8. The MEG apparatus of claim 5, further comprising a photodetector arranged to receive the second light signal from the second end of the optical fiber and to generate an optical measurement signal responsive to detecting the second light signal.

9. The MEG apparatus of claim 8, wherein the photodetector is configured to detect light having a wavelength of approximately 630 nm.

10. The MEG apparatus of claim 9, further comprising an analog-to-digital converter coupled to the photodetector to receive the optical measurement signal.

11. The MEG apparatus of claim 9, further comprising a microprocessor, wherein the microprocessor is configured to analyze the optical measurement signal to determine characteristics of a magnetic field to which the MEG apparatus is exposed.

12. The MEG apparatus of claim 1, further comprising a microwave field generator coupled to an end of the first microwave transmission line and configured to generate the first microwave field.

13. The MEG apparatus of claim 12, further comprising a microwave field control circuit coupled to the microwave field generator to provide the microwave field generator with a microwave source signal, wherein the microwave field control circuit is configured to output the microwave source signal at a frequency between about 50 MHz and about 4 GHz.

14. The MEG apparatus of claim 1, further comprising a plurality of additional leads and a plurality of additional magnetic field sensors attached to the plurality of additional leads, respectively, wherein each additional magnetic field sensor of the plurality of additional magnetic field sensors comprises a corresponding substrate, and a corresponding electron spin defect layer on the substrate of the additional magnetic field sensor, the corresponding electron spin defect layer comprising at least one corresponding lattice defect, wherein a corresponding first spin energy level of the at least one corresponding lattice defect splits upon exposure to a corresponding microwave field.

15. The MEG apparatus of claim 1, comprising a cranial cap, wherein the lead is attached to the cranial cap.

16. An intracranial magnetoencephalography (MEG) device comprising:
a base;
a first intracranial needle comprising a first end attached to the base, wherein the first intracranial needle comprises a first microwave transmission line and a first optical waveguide; and
a magnetic field sensor attached to a second end of the first intracranial needle, wherein the magnetic field sensor comprises a substrate, and an electron spin defect layer on the substrate, the electron spin defect layer comprising at least one lattice defect, wherein a first spin energy level of the at least one lattice defect splits upon exposure to a microwave field.

17. The intracranial MEG device of claim 16, wherein the first microwave transmission line is arranged to provide a first microwave field to the electron spin defect layer and wherein the first optical waveguide is arranged to provide a first light signal to the electron spin defect layer and to receive a second light signal emitted by the electron spin defect layer.

18. The intracranial MEG device of claim 16, wherein the base comprises an optical light source positioned to provide light into the first optical waveguide.

19. The intracranial MEG device of claim 18, wherein the optical light source comprises a light emitting diode or a laser.

20. The intracranial MEG device of claim 18, wherein the optical light source is configured to emit a first light signal having a wavelength of approximately 532 nm.

21. The intracranial MEG device of claim 16, wherein the base comprises a photodetector positioned to receive light from the first optical waveguide and to generate an optical measurement signal responsive to detecting the light from the first optical waveguide.

22. The intracranial MEG device of claim 21, wherein the photodetector is configured to detect light having a wavelength of approximately 630 nm.

23. The intracranial MEG device of claim 21, wherein the base further comprises an analog-to-digital converter coupled to the photodetector to receive the optical measurement signal.

24. The intracranial MEG device of claim 21, wherein the base comprises a microprocessor configured to analyze the optical measurement signal to determine characteristics of a magnetic field to which the MEG is exposed.

25. The intracranial MEG device of claim 24, wherein the base comprises a transceiver configured to emit and receive wireless signals.

26. The intracranial MEG device of claim 16, wherein the base comprises a microwave field generator configured to generate a first microwave field, and wherein the microwave field generator is coupled to the first microwave transmission line.

27. The intracranial MEG device of claim 26, wherein the base further comprises a microwave field control circuit coupled to the microwave field generator to provide the microwave field generator with a microwave source signal, wherein the microwave field control circuit is configured to output the microwave source signal at a frequency between about 50 MHz and about 4 GHz.

28. The intracranial MEG device of claim 16, wherein the first optical waveguide comprises an optical fiber.

29. The intracranial MEG device of claim 16, wherein the electron spin defect layer comprises diamond.

30. The intracranial MEG device of claim 29, wherein the at least one lattice defect comprises a nitrogen vacancy defect.

31. The intracranial MEG device of claim 16, wherein the magnetic field sensor further comprises a bias magnet.
